(19) United States
(12) Patent Application Publication
(10) Pub. No.: US 2019/0231793 A1
(43) Pub. Date: Aug. 1, 2019

(54) USE OF HARRINGTONINES IN THE TREATMENT OF BREAST CANCER, IN PARTICULAR TRIPLE-NEGATIVE BREAST CANCER

(71) Applicant: Jean-Pierre Robin, Martigny (CH)

(72) Inventors: Jean-Pierre Robin, Martigny (CH); Nina Radosevic, Clermont Ferrand (FR); Frédérique Penault-Llorca, Ceyrat (FR)

(73) Assignee: Jean-Pierre Robin, Martigny (CH)

(21) Appl. No.: 16/335,747

(22) PCT Filed: Sep. 22, 2017
(86) PCT No.: PCT/EP2017/074135

§ 371 Date: Mar. 22, 2019

(30) Foreign Application Priority Data
Sep. 22, 2016 (FR) 1658923

(51) Int. Cl. A61K 31/55; A61P 35/00; A61K 9/00

(52) U.S. Cl. CPC A61K 31/55; A61K 9/0053; A61K 9/0019; A61P 35/00

(57) ABSTRACT

The present invention relates to harringtonine, homoharringtonine and/or derivatives thereof, in particular compounds of formula 1a to 1g as defined in table 1, more particularly in the form of salt, preferably crystalline salt, for use in the treatment or prevention of breast cancer, in particular triple-negative breast cancer (TNBC).

### FIELD OF THE INVENTION

The present invention relates to harringtonine, homoharringtonine and/or their derivatives including, especially the compounds of formula (1a) to (1g) as defined in Table 1, more particularly in salt form, preferably crystalline, for use in the treatment or prevention of breast cancer, in especially Triple Negative Breast Cancer (TNBC).

### BACKGROUND

Harringtonines (1) are esters of cephalotaxine (2), alkaloids which exhibit cytotoxic properties, and occurring in most Asian coniferous species belonging to the genus Cephalotaxus. Two of these alkaloids harringtonine (1a) and homoharringtonine (1b) are used mainly in China in the therapy of leukemias.

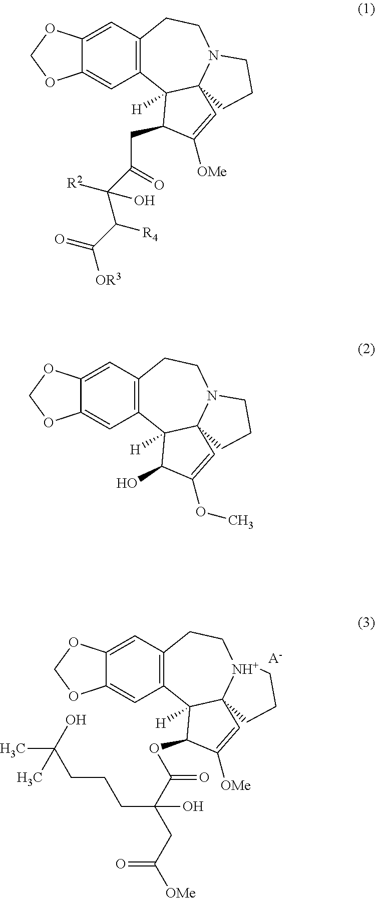

TABLE 1
THE MAIN HARRINGTONINES

| # | Trivial name | R₂ | R₃ | R₄ | Activity |
|---|---|---|---|---|---|
| (1a) | harringtonine | (CH₃)₂COH—(CH₂)₂— | Me | H | anticancer |
| (1b) | homoharringtonine | (CH₃)₂COH—(CH₂)₃— | Me | H | anticancer |
| (1c) | norharringtonine | (CH₃)₂COH—CH₂— | Me | H | none |
| (1d) | deoxyharringtonine | (CH₃)₂CH—(CH₂)₂— | Me | H | anticancer |
| (1e) | bishomoharringtonine | (CH₃)₂COH—(CH₂)₄— | Me | H | none |
| (1f) | isoharringtonine | (CH₃)₂CH(CH₂)₂— | Me | OH | none |
| (1g) | neoharringtonine | C₆H₆—CH₂— | Me | H | cytotoxic |
| (1) | harringtonines | R₂— | Me | R₄ | N/A |
| (1x) | harringtoids | R₂— | R₃ | R₄ | cytotoxic |

Semi-synthetic analogs such as (1x) exhibited also promising results. Homoharringtonine (1b) or HHT (named also omacetaxine mepesuccinate as an Active Ingredient, is now approved in the United States in the treatment of chronic myeloid leukemia which failed to respond to Tyrosine Kinase Inhibitor. A version produced by semi-synthesis [Robin et al. Tetrahedron Letters 1999, 40, 15, p 2931 “The First Semi-synthesis of Enantiopure Homoharringtonine via Anhydrohomoharringtonine from a Preformed Chiral Acyl Moiety”] of this molecule is currently marketed in the United States. HHT is also used in the therapy of Acute Myeloid Leukemia, experimentally in the Western countries and routinely in China, its country of origin. This anticancer agent and one of its congeners, harringtonine (HA) (1a) are also used with some effectiveness in other hematosarcomas such as, for example, myelodysplastic syndrome and Polycytemia Vera, and, experimentally, in multiple myeloma. In most cancers (i.e. solid tumors), this inhibitor of protein synthesis has not yielded conclusive results so far. However, most clinical studies date back to a time when the toxicity, due to the poor quality of this product, had not yet been mastered. Among these studies, the respond to mammary tumors in general, both in vitro and in vivo, had been poor or null at all in vivo [Ajani et al. Cancer Treatment Report 1986, p 376 “Phase II studies of homoharringtonine in patients with advanced malignant melanoma; sarcoma; and head and neck, breast, and colorectal carcinomas”].

The development of new technologies for both synthesis and purification has significantly changed this situation: medicine now has unlimited quantities of very pure semi-synthetic or natural versions of this product. A version in the form of crystalline organic salts [for example (3)] easily purifiable and directly soluble in aqueous media has even been described just recently. Triple-negative breast cancer (TNBC) is characterized by the absence of the only 3 receptors that currently allows the targeted treatment of this cancer: estrogen receptor, progesterone receptor and Human Epidermal growth factor Receptor 2 (HER2). Thus, the treatment of TNBC, most of which are aggressive cancers, is based on pre-operative chemotherapy, called neoadjuvant (NACT), based on the standard anthracycline-taxane combination. The disappearance of invasive tumor cells in the breast and lymph nodes (called pathological complete response, pCR) after this treatment is associated with prolonged survival of patients. Nevertheless, pCR is observed in only 20 to 35% of patients [Liedke et al. J Clin Oncol. 2008 Mar. 10; 26(8):1275-81]. The presence of the residual tumor (without pathological complete response, npCR) after NACT carries a significant risk of developing visceral metastases (brain, lung, liver). The survival of patients with a metastatic TNBC is only 12 to 18 months [Sharma Oncologist. 2016 September; 21(9):1050-62] because during this phase the disease presents an increased resistance to the current therapeutic armamentarium.

It is therefore urgent to find new therapeutic approaches for TNBC, both in neoadjuvant and metastatic situations.

The major axis of cell survival, the PI3K-AKT-mTOR signaling pathway, is over-activated in 50-70% of the TNBCs, due to alterations present in the genes for PTEN, PI3K and/or AKT [Millis et al. Clin Breast Cancer. 2015 December; 15(6):473-481]. One of the final results of this overactivation is the increase in RNA translation, or the increase in protein synthesis, as shown in FIG. 1. The increased protein synthesis is the basis of at least three of the hallmarks of cancer, particularly for resistance to cell death, invasion/metastasis, and neoangiogenesis (FIG. 2).

The inhibitors of many molecules belonging to the PI3K-AKT-mTOR axis are in development for various cancers but, until now, none is integrated in the treatment of the TNBC. These inhibitors often demonstrate a temporary anti-cancer effect, followed by the development of resistance. In addition, these inhibitors often exhibit significant in vivo toxicity, limiting their clinical use.

HHT as a protein synthesis inhibitor binds to site A of the 60S unit of ribosomes, inhibits tRNA access and prevents the formation of the first peptide bond of the nascent protein chain. Thus, HHT is a protein synthesis inhibitor or RNA translation inhibitor [Tujebajeva et al. Biochim Biophys Acta. 1992 Jan. 6; 1129(2):177-82, Gürel et al. J Mol Biol. 2009 May 29; 389(1):146-56], and it is the only protein synthesis inhibitor currently available on the market as an anticancer agent.

### BRIEF DESCRIPTION OF THE INVENTION

The present invention has revealed, surprisingly, a significant activity of harringtonines not only on breast cancer strains untreatable until now, but also on so-called triple negative cancers.

For the purposes of the present invention, triple-negative breast cancer (TNBC) is characterized by the absence of the only 3 receptors that currently allow the targeted treatment of this cancer: Estrogen Receptor, Progesterone Receptor and Human Epidermal growth factor Receptor 2 (HER2).

Knowing that a large portion of the TNBCs can exhibit increased protein synthesis, due to PI3K-Akt-mTOR axis abnormalities, the inventors have demonstrated the efficacy and mechanism of action of harringtonines on TNBC cell lines.

The present invention therefore relates to harringtonine, homoharringtonine and/or its derivatives, in particular the compounds of formula 1a to 1g as defined in Table 1, for use in the treatment or prevention of breast cancer, in particular triple-negative breast cancer (TNBC).

The present invention also relates to harringtonine, homoharringtonine and/or their derivatives, in particular the compounds of formula 1a to 1g as defined in Table 1, for use as inhibitors of the proliferation of cancer cell lines CAL-51, MDA-MB-157, MDA-MB-468 et/ou MDA-MB-231.

The subject of the present invention is also a pharmaceutical composition comprising, as active principle, harringtonine, homoharringtonine and/or its derivatives, in particular the compounds of formula 1a to 1g as defined in Table 1, with at least one pharmaceutically acceptable excipient for use in the treatment or prevention of breast cancer, in particular triple negative breast cancer (TNBC).

The present invention also relates to a kit comprising:
(a) a first composition comprising, as active principle, harringtonine, homoharringtonine and/or its derivatives, in particular the compounds of formula (1a) to (1g) as depicted in Table 1, with at least one pharmaceutically acceptable excipient, and,
(b) a second composition comprising an additional therapeutic agent, especially useful in cancer treatment, typically selected from the group consisting of a chemotherapeutic or antiproliferative agent, as a combination product for separately, concomitantly or sequentially use for use in the treatment of breast cancer, in particular triple negative breast cancer (TNBC)

### Definitions

Homoharringtonine has the formula:

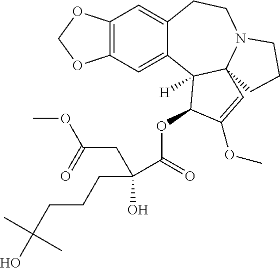 [SMILES: COC(=O)C[C@](O)(CCCC(C)(C)O)C(=O)O[C@@H]1C(OC)=C[C@]23CCCN2CCc2cc4c(cc2[C@H]13)OCO4]

In the field of cancer, the definition of the term “cytotoxicity” is the toxicity for tumor cells in culture; the definition of the term “antitumor” is “in vivo” activity in experimental systems; and the term “antineoplastic” or “anticancer” is reserved for data obtained in clinical trials.

The molecule (1a) is the first cephatotaxine ester isolated from Cephalotaxus harringtonia.

The prefix “homo” in homoharringtonine, also called omacetaxine as an active pharmaceutical ingredient, means that molecule (1b) has one more carbon atom than harringtonine (1a) in its side-chain.

The prefix “nor” in “norharringtonine” means that the molecule (1c) contains one atom less than harringtonine (1a) in its side chain.

In the present invention, the term “pharmaceutically acceptable” is intended to mean that which is useful in the preparation of a pharmaceutical composition which is generally safe, non-toxic and neither biologically nor otherwise undesirable and which is acceptable for veterinary as well as human pharmaceutical use.

In the present invention, the term “pharmaceutical composition” refers to any composition consisting of an effective dose of a compound of the invention and at least one pharmaceutically acceptable excipient. Such excipients are selected, depending on the pharmaceutical form and the desired method of administration, from the excipients usually known to those skilled in the art.

The term “pharmaceutically acceptable salts” of a compound means salts which are pharmaceutically acceptable, as defined herein, and which possess the desired pharmacological activity of the parent compound. Such salts generally include:
(1) hydrates and solvates,
(2) pharmaceutically acceptable acid addition salts formed with pharmaceutically acceptable inorganic acids such as hydrochloric acid, hydrobromic acid, sulfuric acid, nitric acid, phosphoric acid and the like; or formed with pharmaceutically acceptable organic acids such as acetic acid, benzenesulfonic acid, benzoic acid, camphorsulfonic acid, citric acid, ethanesulfonic acid, fumaric acid, glucoheptonic acid, gluconic acid, glutamic acid, glycolic acid, hydroxynaphthoic acid, 2-hydroxyethanesulfonic acid, lactic acid, maleic acid, malic acid, mandelic acid, methanesulfonic acid, muconic acid, 2-naphthalenesulfonic acid, propionic acid, salicylic acid, succinic acid, dibenzoyl-L-tartaric acid, tartaric acid, p-toluenesulphonic acid, trimethylacetic acid, trifluoroacetic acid and the like, but also the salts of malonic acid and tartronic acid, or,
(3) pharmaceutically acceptable base addition salts formed when an acidic proton present in the parent compound is either replaced by a metal ion, for example an alkali metal ion, an alkaline earth metal ion or an aluminum ion; or is coordinated with a pharmaceutically acceptable organic or inorganic base. Acceptable organic bases include diethanolamine, ethanolamine, N-methylglucamine, triethanolamine, tromethamine and the like. Acceptable inorganic bases include aluminum hydroxide, calcium hydroxide, potassium hydroxide, sodium carbonate and sodium hydroxide.

Typically, these salts include (1) hydrates and solvates, and (2) pharmaceutically acceptable acid addition salts as listed above in point (2).

In the present invention, the term “treatment” applies to all types of animals, preferably to mammals, and more preferably to humans.

In the case of treatment of a non-human animal, the term will refer to veterinary treatment. However, in the present invention, the main focus is on the treatment of breast cancer in woman.

### DETAILED DESCRIPTION OF THE INVENTION

The present invention thus relates to harringtonine, homoharringtonine and/or its derivatives, in particular the compounds of formula 1a to (1g) as depicted in Table 1, for use in the treatment or prevention of breast cancer, especially, the triple-negative breast cancer (TNBC).

Preferably, the present invention relates to homoharringtonine for use in the treatment or prevention of breast cancer, in particular triple negative breast cancer (TNBC).

Harringtonine, homoharringtonine and/or its derivatives, in particular the compounds of formula (1a) to (1g) as defined in Table 1, preferably homoharringtonine, especially in salt form, advantageously crystalline salt, are used as inhibitors proliferation of cancer cell lines CAL-51, MDA-MB-157, MDA-MB-468 and/or MDA-MB-231.

Harringtonine, homoharringtonine and/or its derivatives, in particular the compounds of formula (1a) to (1g) as defined in Table 1, preferably homoharringtonine, especially in the salt form, advantageously of crystalline salt, can be used to prepare pharmaceutical compositions comprising, as active principle, harringtonine, homoharringtonine and/or its derivatives, in particular the compounds of formula (1a) to (1g) as defined in Table 1, preferably homoharringtonine, especially in the form of salt, advantageously of crystalline salt, with at least one pharmaceutically acceptable excipient. Thus, the present invention relates to a pharmaceutical composition comprising harringtonine, homoharringtonine and/or its derivatives, especially the compounds of formula 1a to (1g) as defined in Table 1, preferably homoharringtonine, especially, in the form of salt, preferably crystalline salt, as active ingredient, and a pharmaceutically acceptable excipient.

Said excipients are selected according to the pharmaceutical form and the desired mode of administration from the usual excipients which are known to those skilled in the art.

Harringtonine, homoharringtonine and/or its derivatives, in particular the compounds of formula 1a to (1g) as defined in Table 1, preferably homoharringtonine, especially in salt form, advantageously of crystalline salt, or the compositions of the invention are therefore useful for the treatment or prevention of breast cancer, including triple-negative breast cancer (TNBC).

Advantageously, harringtonine, homoharringtonine and/or its derivatives, especially the compounds of formula (1a) to (1g) as defined in Table 1 are in the form of salts, such as inorganic or organic salts, preferably crystalline. Such salts are, for example, obtained as described in application WO 2015/101628. Examples of salts include the addition salts with fumaric, malic, lactic, tartaric, citramalic, itaconic, succinic, maleic, malonic, tartaronic, citric, salicylic and hydrochloric acid.

Harringtonine, homoharringtonine and/or its derivatives, especially the compounds of formula (1a) to (1g) as defined in Table 1, preferably homoharringtonine, in particular in salt form, advantageously crystalline salt, can be used alone or in combination with an additional therapeutic agent, especially useful in a treatment against cancer, typically selected from the group consisting of a chemotherapeutic or antiproliferative agent, and an agent targeted therapy such as an anti-inflammatory agent an immunomodulatory or immunosuppressive agent, an agent for the treatment of a neurological disorder an agent for treating a cardiovascular disease an agent for the treatment of destructive bone disorders, an agent for treating a liver disease, an anti-viral agent, an agent for treating blood disorders, an agent for the treatment of diabetes, an agent for the treatment of immunodeficiency disorders and/or an agent for the treatment of pain.

The present invention therefore also relates to a kit comprising:
(a) a first composition comprising harringtonine, homoharringtonine and/or its derivatives, in particular the compounds of formula (1a) to (1g) as defined in Table 1, preferably homoharringtonine, especially in salt form, advantageously of crystalline salt, as an active ingredient, and a pharmaceutically acceptable excipient, and
(b) a second composition comprising a therapeutic additional agent, especially useful in cancer treatment, typically selected from the group consisting of a chemotherapeutic or antiproliferative agent, as a combination product for separately, concomitant or sequentially use.

Said kit is useful as a medicament, particularly for treating breast cancer, in particular triple negative cancer (TNBC)

The pharmaceutical compositions according to the invention may be administered parenterally, such as intravenously, intraarterially, intrathecally, intratumorally or intradermally, or topically, orally or nasally. Parenteral dosage forms include aqueous suspensions, isotonic saline solutions or sterile and injectable solutions which may contain pharmacologically compatible dispersing agents and/or wetting agents. Orally administrable forms include tablets, soft or hard capsules, powders, granules, oral solutions and suspensions. Nasal administrable forms include aerosols. Topically administrable forms include patches, gels, creams, ointments, lotions, sprays, eye drops.

Preferably, the compounds or compositions of the invention are administered orally or parenterally (especially subcutaneously or intravenously).

The effective dose of a compound of the invention varies according to many parameters such as, for example, the chosen route of administration, weight, age, sex, the progress of the pathology to treat and the sensitivity of the individual to treat.

The present invention, in another aspect, also relates to a method of treating breast cancer, in particular triple negative breast cancer (TNBC), which comprises administering to a patient in need an effective dose of harringtonine, homoharringtonine and/or its derivatives, in particular the compounds of formula (1a) to (1g) as defined in Table 1, preferably of homoharringtonine, in particular in the form of salt, advantageously of crystalline salt, or of a composition according to the invention, preferably parenterally (especially subcutaneously or intravenously) or orally.

### DESCRIPTION OF THE FIGURES

FIG. 1. Diagram showing the PI3K-AKT-mTOR signaling pathway.

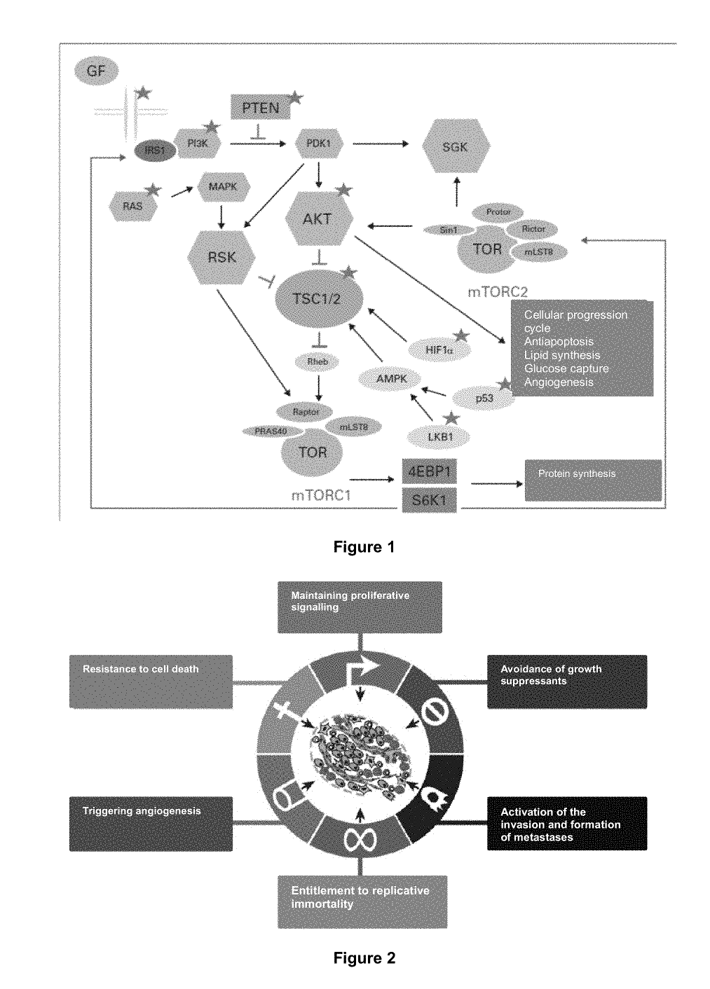

FIG. 2. Diagram showing the mechanisms implemented by cancer cells that allow their resistance and proliferation.

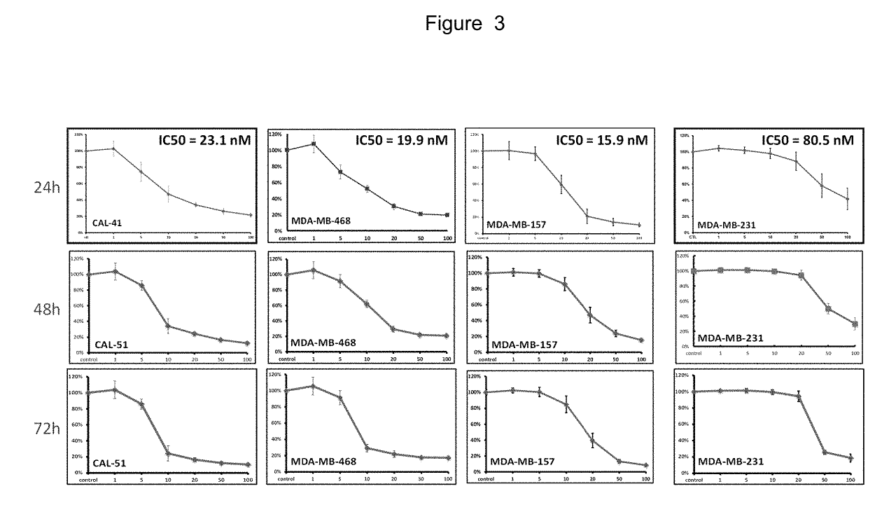

FIG. 3. Inhibition of cell proliferation by HHT X-axis: concentration (nM); Y axis: viable fraction (Fv=% of viable cells). HHT significantly reduced, after only 24 hours, the proliferation of 3 out of 4 lines analyzed (IC50 15.9-21.3 20 nM). A comparable effect on MDA-MB-231 was observed at higher concentrations (IC50 80.5 nM). In addition, by prolonging the incubation time, the inhibition of the proliferation of MDA-MB-231 was significantly increased (Fv at 50 nM: 58.1±14.5% vs 25.9±2.4%, 24 h vs. 72 h resp., P<0.01; Fv at 100 nM: 41.7±13.3% vs. 18.7±4.8%, 24 h vs. 72 h resp., P<0.01). The effect of HHT on the proliferation of CAL-51, MDA-MB-468 or MDA-MB-157 was independent of the incubation time, regardless of the concentrations (p>0.05).

FIG. 4. Effect of HHT on the cell cycle. After 48 hours of exposure to increasing concentrations of HHT, the accumulation of CAL-51 and MDA-MB-157 cells in Set GO/G1 phase was observed at 20 nM and 50 nM, respectively. The MDA-MB-468 or MDA-MB-231 cells were stopped in the G2/M phase with 20 nM or 50 nM HHT, and in the S phase with 100 nM of the product. From the bottom up on each bar of the histogram: GOG1, S-Phase, and possibly G2M.

FIG. 5. Effect of HHT on apoptosis. HHT induces apoptosis of CAL-51, MDA-MB-468 and MDA-MB-157 cells in a concentration and time dependent manner, while in MDA-MB-231 cells no significant apoptosis was observed, regardless of the concentration of HHT or the incubation time. Title Translation: % of cells in early and late apoptosis

FIG. 6. Effect of HHT on the expression of apoptosis regulatory proteins. The tests were conducted at a concentration of 100 nM HHT. As already published in the case of leukemic cells [Tang et al. Mol Cancer Ther. 2006 March; 5 (3): p 723], HHT induces, in all lines analyzed except MDA-MB-231, a very fast and strong drop in the level of Mcl-1, one of the antiapoptotic key proteins. The same effect was observed for Bcl-2, in line MDA-MB-468, and for survivin, in CAL-51. The reduction in the XIAP rate was more modest and occurred later. The reduction in Mcl-1 was observed before the reduction in total PARP1 (PARP1-t) or total caspase-3 (caspase 3-t), and before the appearance of cleaved PARP-1, produced during apoptosis. We conclude that HHT induces apoptosis of CAL-51, MDA-MB-468 and MDA-MB-157 cells by reducing the level of anti-apoptotic proteins, probably by inhibiting their synthesis. On the other hand, no effect of HHT on the level of these proteins was observed in line MDA-MB-231, which corresponds to the absence of apoptosis of this line.

FIG. 7. Typical example of the effect of HHT on MDA-MB-231 triple negative breast tumor xenograft. 7a: control (7 control mice), abscissa: control mice (7), ordinate: volume of the xenograft in mm3. 7b: group treated with HHT (group of 8 Total of 20 mice, abscissa: treated mice (8), ordinate: xenograft volume in mm3.

### EXAMPLES

The invention will be better understood on reading the following examples, which are given purely by way of illustration and should not be interpreted as limiting the scope of the present invention.

### Example 1: In Vitro Study Method

Cell Lines: CAL-51 (PIK3CA mutated) and MDA-MB-157 (without apparent genomic alteration of the PI3K-AKT-mTOR axis (NF1, TP53 mutated) were cultured in DMEM+10% FCS+200 μg/ml gentamicin, in humidified atmosphere, at 5% CO 2.

MDA-MB-468 (PTEN, RB1, SMAD4, mutated TP53, amplified EGFR/ERBB1) and MDA-MB-231 (highly metastatic, without alteration of PI3K-Akt-mTOR; KRAS, BRAF, CDKN2A, PDGFRA, (NF2, Mutated TP53) were cultured in Liebowitz's medium+10% FCS+200 μg/ml gentamycin, in a CO2-free atmosphere

Drug: commercial HHT (Sigma-Aldrich), purified by recrystallization according to Liu's technique [U.S. Pat. No. 62,073, 14 May 1987], was provided free of charge by LeukePharma (Le Mans, France). The stock solution of 20 mM HHT citrate was obtained by dissolution in citric acid (Sigma-Aldrich), in equimolar amount or by direct dissolution of the crystalline homoharringtonine salt in sterile purified water. The following dilutions of this stock solution were performed using sterile PBS buffer. All of the solutions was stored at −80° C.

Cell viability: The cells were seeded in the 96-well plates at the concentration of 5000 cells/well (3.3×104/ml) for the MDA-MB-lines, and at the concentration of 1000 cells/well (6.6×10 3 ml) for CAL-51. After 24 hours of rest, at the confluence of 50-60%, the cells were treated with increasing concentrations of HHT for 24 h, 48 h and 72 h. After washing twice with sterile PBS and 48 h rest, the cells were fixed with trichloroacetic acid. After the addition of sulforhodamine blue (SRB), the absorbance was measured at 540 nm (Multiskan™ FC plate photometer, Thermo Scientific). IC50s were calculated by CompuSyn (Chou & Talalay, ComboSyn Inc., Paramus, N.J., USA).

Cell cycle and apoptosis: the number of cells in different phases of the cell cycle or in apoptosis was determined by flow cytometry using FITC Annexin V Apoptosis Detection Kit 1 (BD Biosciences) and following the manufacturer's recommendations. Briefly, 3×10 4 cells of each line were seeded in 6-well plates and, after 24 h resting, treated with increasing concentrations of HHT for 6-72 h, then washed and incubated with propidium iodide and or with FITC annexin V). The fluorescence was measured using a BD LSH II cytofluorometer (BD Biosciences).

Western blot: 5×10 5 cells, in 10 ml of medium, were inoculated in the 100 mm Petri dishes. After 24 hours of rest, the cells were incubated with 100 nM of HHT for different times, staggered for 2 hours and 48 hours. Then, the cells were washed and lysed directly into the dishes (without trypsinization) using RIPA buffer (Pierce-ThermoScientific), containing a mixture of protease inhibitors (Sigma). The protein concentration was determined by the method of Bradford. Protein electrophoresis was performed in 12-30% polyacrylamide gels. After transfer to the PVDF membrane, the revelation was carried out by ECL Plus (Amersham).

### Example 2

Using the methods described in Example 1, homoharringtonine 1b was tested on line CAL-51 (mutated PIK3CA).

### Example 3

Using the methods described in Example 1, harringtonine 1a was tested on line CAL-51 (mutated PIK3CA).

### Example 4

Using the methods described in Example 1, homoharringtonine 1b was tested on line MDA-MB-157.

### Example 5

Using the methods described in Example 1, homoharringtonine 1b was tested on line MDA-MB-458.

### Example 6

Using the methods described in Example 1, homoharringtonine 1b was tested on the MDA-MB-231 line.

### Example 7

In vivo study of HHT on TSTN MDA-MB-231

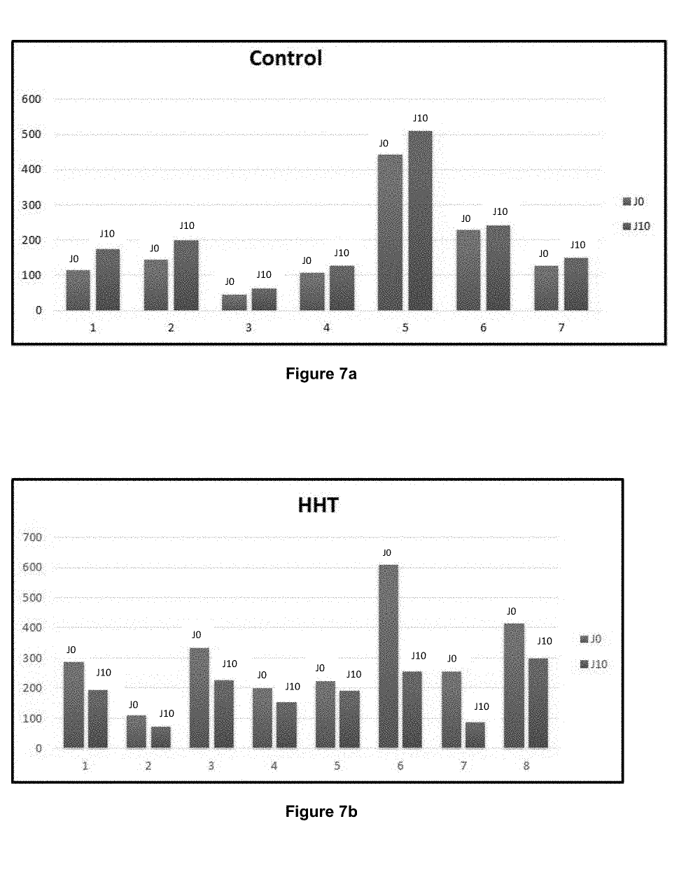

The efficacy of homoharringtonine in vivo on xenografts of the triple-negative breast cancer line (MDA-MB-231), was performed on Swiss nudes immuno-depressed mice (nu/nu), 10 years old. −12 weeks. The tumor was grafted in 16 animals, by injecting 5×10 6 cells of the tumor strain, subcutaneously in the dorsal region, until a tumor volume of 200-300 mm 3 was obtained. 8 mice received 1 mg of HHT (as salt) per kg subcutaneously twice daily for 8 days. The control group (7 mice) received only physiological saline in the same dosage as HHT. Mice were sacrificed 3 days after stopping treatment, recovered xenografts, and measured tumor size. The tissues were then formalin-fixed and paraffin-embedded for subsequent histological analyzes. As shown in FIGS. 7a and 7b, the difference in tumor volume between OJ and J10 was noted. A blatant decrease (20 to 60%) in tumor volume was noted in the lot of animals treated, whereas an increase (20-30%) of said tumor volume was noted in the control group.

### Example 8: In Vivo Study of Harringtonine on TSTN MDA-MB-231

Based on the method used in Example 7 but using harringtonine (1a) at doses of 2 mg per Kg similar results were obtained.

### Example 9

Using the methods described in Example 7, homoharringtonine (1b) was tested on line CAL-51 (mutated PIK3CA).

### Example 10

Using the methods described in Example 7, harringtonine (1a) was tested at a dose of 2 mg/kg on the CAL-51 line (mutated PIK3CA).

### Example 11

Using the methods described in Example 7, homoharringtonine (1b) was tested on line MDA-MB-157.

### Example 12

Using the methods described in Example 7, homoharringtonine (1b) was tested on line MDA-MB-458.

### CONCLUSION

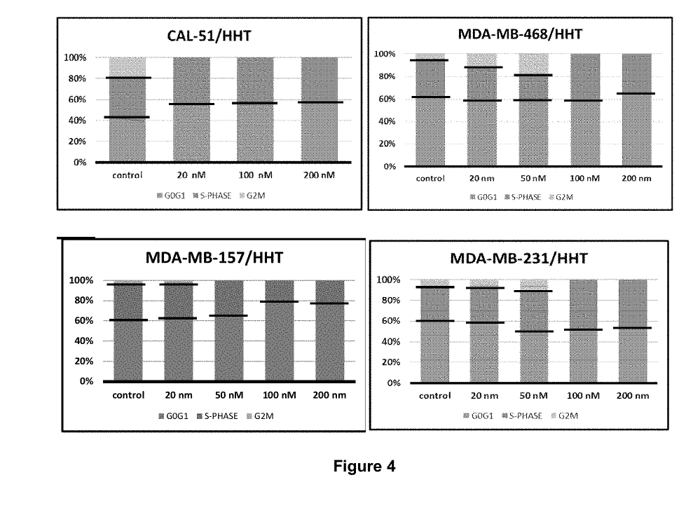

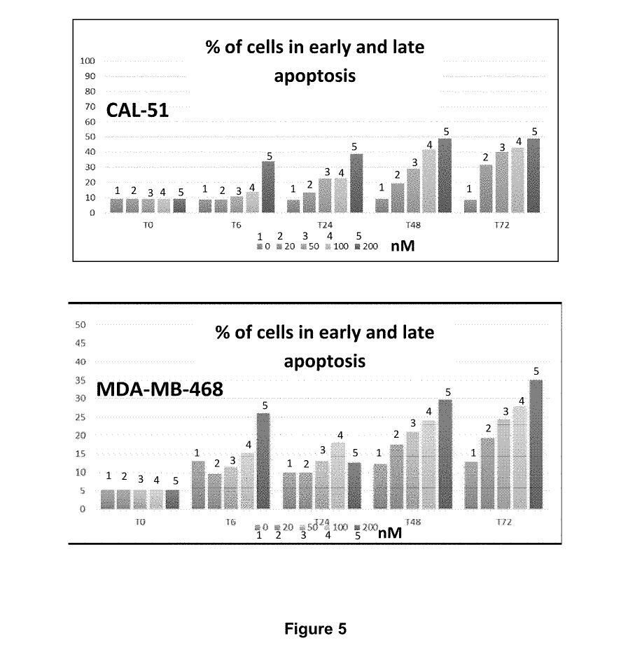

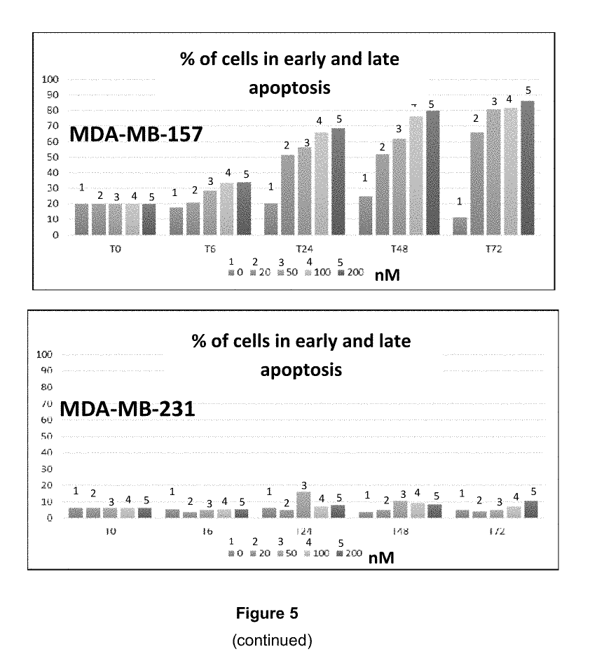

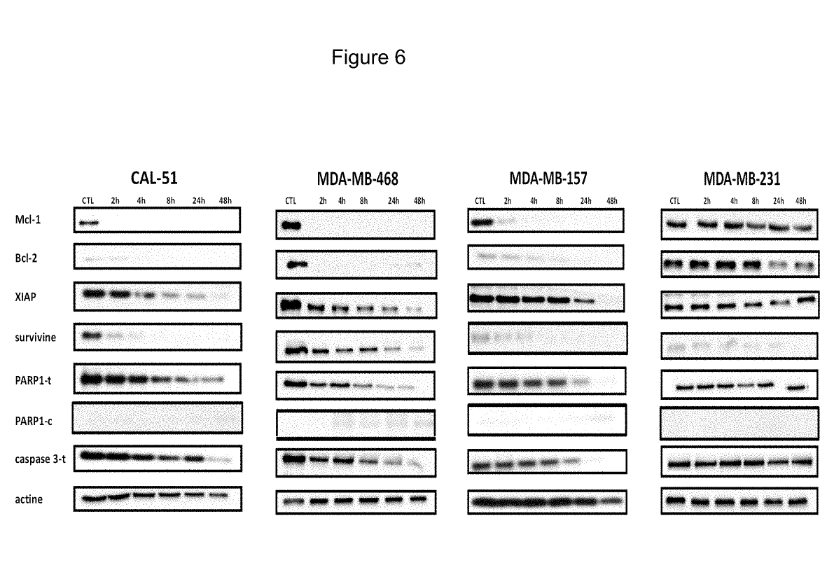

The results obtained in Examples 2 to 12 are described in FIGS. 3 to 7.

This study is the first to demonstrate that HHT induces in vitro apoptosis and/or cytostasis of TNBC cell lines and, in vivo reduction of tumor volume on TNBC strain-grafted tumors. The likely mechanism of action of HHT is the inhibition of short half-life anti-apoptotic protein synthesis, such as Mcl-1, Bcl-2, and/or survivin. Thus, removal of the anti-apoptotic brake quickly triggers apoptosis of cells highly sensitive to HHT. This effect may potentiate the apoptotic action of anti-cancer drugs currently used in the treatment of TNBC. Studies of HHT associations and such drugs are underway.

1. Homoharringtonine for use in the treatment or prevention of breast cancer.

2. Homoharringtonine for use according to claim 1, characterized in that the breast cancer is triple-negative breast cancer (TNBC).

3. Homoharringtonine for use according to claim 1 or 2, characterized in that the homoharringtonine is in salt form.

4. Homoharringtonine for use as inhibitors of proliferation of cancer cell lines CAL-51, MDA-MB-157, MDA-MB-468 and/or MDA-MB-231.

5. Homoharringtonine for use according to claim 4, characterized in that the homoharringtonine is in salt form.

6. Homoharringtonine for use according to claim 3 or 5, characterized in that the homoharringtonine salt is an addition salt with fumaric, malic, lactic, tartaric, citramalic, itaconic, succinic, maleic, malonic, tartronic acid. citric, salicylic and hydrochloric.

7. Pharmaceutical composition comprising as active principle homoharringtonine, with at least one pharmaceutically acceptable excipient, for use in the treatment or prevention of breast cancer, in particular breast cancer is “triple-negative” breast cancer (TNBC).

8. Pharmaceutical composition for use according to claim 7, characterized in that the homoharringtonine is in salt form, in particular an addition salt with fumaric acid, malic acid, lactic acid, tartaric acid, citramalic acid, itaconic acid, succinic acid, maleic acid, malonic, tartronic, citric, salicylic and hydrochloric.

9. Kit comprising:
(a) a first composition comprising as active principle homoharringtonine, with at least one pharmaceutically acceptable excipient, and
(b) a second composition comprising an additional therapeutic agent, especially useful in a treatment against cancer, typically selected from the group consisting of a chemotherapeutic or antiproliferative, as a combination product for use separately, concomitant or sequentially over time to use for the treatment of breast cancer, in particular triple negative breast cancer (TNBC).

10. Kit for use according to claim 9, characterized in that the homoharringtonine is in salt form, especially an addition salt with fumaric acid, malic, lactic, tartaric, citramalic, itaconic, succinic, maleic, malonic, tartronic, citric, salicylic and hydrochloric.
